(19) United States
(12) Patent Application Publication
(10) Pub. No.: US 2015/0119908 A1
(43) Pub. Date: Apr. 30, 2015

(54) EXTRAVASCULAR DEVICES SUPPORTING AN ARTERIOVENOUS FISTULA

(71) Applicant: Abbott Cardiovascular Systems Inc., Santa Clara, CA (US)

(72) Inventors: Paul Consigny, San Jose, CA (US); Stephen D. Pacetti, San Jose, CA (US); William E. Webler, JR., San Jose, CA (US)

(73) Assignee: Abbott Cardiovascular Systems Inc., Santa Clara, CA (US)

(21) Appl. No.: 14/063,984

(22) Filed: Oct. 25, 2013

(51) Int. Cl. A61B 17/11

(52) U.S. Cl. CPC A61B 17/11; A61B 2017/1107; A61B 2017/1135
USPC 606/156

(57) ABSTRACT

A medical device includes a curved tubular body configured for being used as an extravascular device to support vein maturation following the formation of an arteriovenous fistula. The tubular body is curved. The tubular body has an entrance angle of less than about 40 degrees to improve blood flow from the artery into the vein. And the tubular body includes a cuff or edge at the proximal end to stabilize the tubular body at the fistula.

### FIELD OF THE INVENTION

The present invention relates to implantable medical devices associated with the creation of, and/or the maturation of an arteriovenous (AV) fistula access structure for hemodialysis.

### BACKGROUND OF THE INVENTION

AV Fistula (a connection between an artery and a vein) are a desired access structure for the dialysis of kidney failure patients. FIG. 1 illustrates a matured portion of the vein near the artery, which acts as a re-usable cannula access site proximal the AV fistula.

About 42% of surgically created AV Fistula fail to mature; that is, the portion of the vein proximal the fistula fails to adapt physiologically to accommodate the higher arterial pressure. When this venous portion (or side of the AV fistula) matures, it becomes usable as a cannula access site for dialysis (FIG. 1). Maturation can take about 6 weeks from forming the fistula. Failure to mature and/or act as a good dialysis access site is most commonly the result of poor blood flow (low blood pressure/low blood flow rates) in the venous portion of the fistula. About 74% of these failures are salvaged by some form of intervention, followed by maturation of the venous side in another 6-8 weeks. The remaining about 11% of the cases are regarded as failures, which necessitates creating an AV Fistula at another site. The most common site of initial AV Fistula creation is the wrist. If a new AV Fistula is required, a new site proximal of the previous/failed site is chosen. Typically, there are 3 potential sites per arm.

Patients without a mature AV Fistula require some other, less desirable form of dialysis access for the standard 3 times a week dialysis regimen until a mature fistula is available. Additionally, about a third of mature fistula fail in a year. The health of kidney failure patients without a functioning mature AV Fistula deteriorates at a more rapid rate than those with one. Deteriorating health makes the subsequent creation of a functioning mature AV Fistula less probable, necessitating a significant number of interventions or access procedures resulting in poorer survival rates. Thus, a significant number of interventions and procedures may be avoided or significantly delayed, significant cost savings realized and the survival rate of dialysis patients significantly improved by decreasing the failure to mature rate of newly created AV fistula and by reducing the rate at which mature fistula fail.

There is evidence that the shape of an arteriovenous fistula can affect long term durability. For example, Papachristou (2012) and Krishnamoorthy (2012) have indicated that a curved fistula is preferred to a straight fistula because the curved fistula results in greater flow rates, lesser differences in wall shear stress, greater venous dilatation, and less eccentric neointimal hyperplasia. Papachristou E and Vazquez-Padron R I. From basic anatomic configuration to maturation success. Kidney International 81: 724-726, 2012. Krishnamoorthy M K, Banerjee R K, Wang Y et al. Anatomic configuration affects the flow rate and diameter of porcine arteriovenous fistula. Kidney International 81: 745-750, 2012. In addition, Ene-lordache B et al (2013) have found that angle at the origin of a “side-to-end” arteriovenous fistula is very important. Their research indicates that an angle of 30 degrees is preferred over angles of 45, 60 or 90 degrees. Ene-lordache B, Cattaneo L, Dubini G, Remuzzi A. Effect of anastomosis angle on the localization of disturbed flow in “side-to-end” fistula for haemodialysis access. Nephrol Dial Transplant 28: 997-1005, 2013.

There are no known extravascular or perivascular devices available that can effectively and reliably assist a surgeon in creating a more desirable AV fistula construct. Accordingly, there is a need for a construct that can aid in creating the correct anatomy by providing the appropriate support in the appropriate locations and in the appropriate configurations that promote long-term arteriovenous (AV) fistula patency.

### SUMMARY OF THE INVENTION

The invention provides an extravascular or perivascular arteriovenous (AV) guide intended for being placed at an anastomosis to support and help achieve vein maturation. The AV guide is configured to maintain a curved shaped for the vein portion of the fistula as it expands in diameter during the maturing process. When the vein portion of the fistula has a curved shape, or there is a more gradual change of direction for blood flow from the artery into the vein, it is believed that the desired maturation of the vein and increased patency is more likely to occur. The AV guide is intended to cause the vein to mature into a shape producing less acceleration of the flow as it is diverted from the artery to vein than if the vein portion were to extend from the artery at a relative high angle (e.g., greater than 45 degrees). With an AV guide according to the invention it is believed there can be less stagnant or circular blood flow in or near a surgically formed AV fistula.

The invention is, in one aspect, directed to medical device forming a curved vein portion of the fistula. In particular, this aspect of invention provides devices and methods to decrease the failure to mature of a created AV Fistula and to reduce the failure rates of mature fistula. Decreasing the failure to mature rate is accomplished by providing devices to aid in the creation of an appropriately angled attachment site or take-off of the vein from the artery. A take-off angle of above about 30 degrees (relative to the artery longitudinal axis, i.e., axis A in FIG. 2D), is associated with low flow (failure) in the fistula. The device(s) may also be designed to help maintain the patency of the attachment site during the maturation period. Loss of (or poor) patency of the attachment site is associated with low flow and eventual failure of the fistula.

Reducing the rate at which mature fistula fail is accomplished by providing an implanted device, referred to herein as an AV guide, to aid in the creation of a controlled curved vein portion from the artery attachment site of the fistula to the undisturbed vein portion. According to some embodiments, the AV guide may have all of the following attributes: the AV guide is installed at the time of surgery to make the AV fistula; the AV guide is installed around the vein, running from the arteriovenous anastomosis up the vein in a proximal direction; the AV guide is installed around the vein, and afterwards the vein is anastomosed to the artery; the AV guide the AV guide has a preformed shape, and radius of curvature; and the AV guide shape is imposed upon the fistula and is designed to increase blood flow and reduce blood flow turbulence.

According to some embodiments, the AV guide is sized to initially fit the vein loosely and has an internal diameter in the range of 4-8 mm. A variety of sizes would be available depending on the patient anatomy. For example, an average outer diameter of a vein fistula is roughly 6 mm, so the AV guide would have an ID of 6 mm.

According to some embodiments, the AV guide includes a bioresorbable or non-bioresorbable scaffold or stent, respectively, which allows a surgeon to more easily create a curved venous fistula. A tubular body (with or without a cuff) is formed. The tubular body may be formed by a network of rings connected by links. The body may be formed from a tube or molded. Alternatively the tubular body may be formed by weaving threads into overlapping helical shapes. Alternatively, the AV guide may be formed of a tubular body covered by a porous material, fabric or weave. The AV guide may have a diameter of 4-10 mm and a length of 5-10 cm; rings and links that allow the tube or tubular portion to take on multiple shapes from linear to curved (e.g., radius of curvature at an inner side of 3-5 mm) and with or without a pre-made taper (larger diameter at distal end than proximal end); a polymer composition that permits part or all of the scaffold to degrade over a 4-8 week period; examples include polycaptrolactone or 50:50 PLGA-PLLA; a slit or slot along the full length of the AV guide (outer side) that is sufficiently large to permit a collapsed vein to be introduced into the lumen of the AV guide; holes (when needed) in the AV guide to enable the passage of sutures that can be used to tie the AV guide in place or tether the AV guide so that it assumes the desired shape and position at the fistula; varying cell sizes with larger cell sizes in the area of the vein (superficial) where the vein is most likely to be punctured with a needle in order to perform dialysis; and drug-eluting (everolimus, sirolimus, paclitaxel or other anti-restenotic drugs or drug combinations).

In some embodiments the surgeon will isolate the artery and vein, join the vein to the artery via an anastomosis, and then place the scaffold around vein distal to the anastomosis. The AV guide may then be reformed into a more desired shape/curve and sutured in place. Thereafter, the skin incision will be closed. In these embodiments the AV guide may have a curvature less than when implanted, or none at all. When sutured in place the tubular body is re-shaped into the desired curve as needed. The AV guide may then take the curved shape having a take-off angle of less than about 30 degrees.

In accordance with the foregoing, there is an AV guide, medical device, method for making such an AV guide, a method of using an AV guide, or method for assembly of a medical device comprising such a AV guide having one or more, or any combination of the following things (1)-(43):
(1) A tubular body formed by braiding, weaving or cutting a tubular body from a pre-made tube.
(2) A perivascular wrap having a shape memory of a tubular body and including a slot, or a tube having no slot. In the case of the wrap, in cross-section (taken perpendicular to a bore axis of the tubular body) the wrap having tubular shape memory has a C-shape (as opposed to the tube, which has a circular shape).
(3) A perivascular wrap having a shape memory of a tubular body covered by a porous material, a fabric or a weave and including a slot, or a tubular body covered by a porous material, a fabric or a weave having no slot.
(4) An AV guide is a wrap that in cross-section (taken perpendicular to a bore axis of the tubular body) has tubular shape memory and is C-shape (as opposed to the tube, which has a circular shape).
(5) The AV guide is a curved tubular body. The tubular body (either a tube or body having shape memory of a tube) has a constant diameter or variable diameter, which increases from a proximal to a distal end thereof. Or the AV guide has a diameter that narrows towards the proximal end.
(6) The AV guide is configured with a curve in one plane (configured with a curve is intended to mean that it is made, manufactured or formed to naturally have a curve in that plane). Or the tube is configured with a curve in two planes, or the tube is configured with a curve in one plane and is capable of being easily deformed when implanted to have a curve in a second plane as well.
(7) An AV guide, placed after a fistula is made, includes a tube-shaped wrap formed by edges, e.g., edges 34, 33 or 34′ and 33′. The edges may be thought of as two portions together forming a tubular-shaped AV guide and being connected to each other through a living hinge along an inner side of the tube-shaped wrap.
(8) The AV guide is curved and tapered, or the central axis extending through the lumen or bore of the AV guide body has a curvature. An inner surface of the AV guide may have a constant curvature, in which case the curvature is defined by a radius of a circle. Or the inner surface curvature may be variable, in which case the curvature at a proximal end of the AV guide is higher than the curvature at a distal end.
(9) An AV guide comprising a hollow, frusto-conical body having a curvature in one plane. Alternatively, the AV guide may be formed of a tubular body covered by a porous material, fabric or weave.
(10) For an AV guide having a tapered diameter, e.g., the diameter at the AV guide opening at the proximal end is less than the diameter of the AV guide at a distal end, the curvature along an outer surface of the AV guide is variable or not a constant curvature. The curvature at the outer surface proximal and distal ends may be less than or greater than the curvature along the outer surface intermediate or between the proximal or distal ends, and/or the curvature at the proximal end may be greater than the curvature at the distal end or the distal end curvature may be greater than the proximal end. The inner curved part may have a constant curvature. In some embodiments the inner curved part may trace about half the circumference of a circle. The outer curved part and inner curved part preferably do not have the same curvature, as the AV guide is preferably made to have a tapered shape in this embodiment.
(11) An inner curved part has a higher curvature (at least at the proximal end) than the outer curved part, as it is desirable to have a low take-off angle θ. For example, the take-off angle may be about 5, 10, 15, 20, 25 or less than about 30 degrees relative to axis A in FIG. 2D or the bore axis of the tubular portion 25 in FIG. 3.
(12) The outer part may have a constant curvature, although different from and less than the curvature of the inner part. Stated differently, the outer part may trace a portion of the circumference of a larger circle than a portion of the circumference of a circle traced by the inner part. When the tubular body is tapered the center of these circles are not coincident.
(13) The AV guide distal opening is oriented at between about 80 to 120 degrees from the AV guide proximal opening, which encompasses at least a portion of the opening cut into the artery to form the anastomosis. Connecting these ends is a curved, tapered, and tubular shaped body.
(14) An AV guide having a luminal and abluminal surface, the luminal surface configured for coming into contact with an abluminal surface of a vein and or artery portion of an AV fistula when the AV guide is placed at the fistula. The AV guide has disposed on its luminal surface a composition including everolimus, sirolimus, paclitaxel or other anti-restenotic drug applied as a coating, impregnated or infused in the AV guide. The drug is located at the proximal end of a tubular portion and/or cuff portion of the AV guide, and/or the drug is located only or substantially along about 10%/20%, 10-30% or 10-50% of the length of the tubular portion of the AV guide including a proximal end nearest the anastomosis.
(15) An open slit or slot extends between the proximal and distal ends of the AV guide. When the slot is present, the slot extends along the outer side of the AV guide. When implanted the outer side may be against exposed tissue under the vein (and in some embodiments under a portion of the artery). In some embodiments, this outer side of the AV guide is fixed to the underlying tissue using sutures or adhesive. Thus, expansion of the vein will be guided to have a nearly circular cross-section.
(16) An open slit or slot extends between the proximal and distal ends of the AV guide. When the slot is present, the slot extends along the outer side of the AV guide. When the AV guide of this embodiment is initially placed, it may have a tubular shape. As the vein matures and presses against walls of the AV guide, it may no longer have a strictly tubular shape if the slot edges are pushed apart from each other.
(17) An open slit or slot is formed as an undulating, sinusoidal, square tooth or zig-zag shape, such that the sides of the slit or slot appear to interlock with each other. As the vein matures and presses against the walls of the AV guide, the vein is maintained in a more tubular shape than if the open slit or slot was a straight shape.
(18) Or a slit or slot of a tubular portion of an AV guide forms a space between edges. This space has a curvilinear form to promote vein maturation whereby the cross-section of the matured vein is circular for a vein portion disposed within the tubular portion.
(19) An open slot or slit has its edges in contact with or very close to each other and the AV guide is opened (i.e., its slot edges moved apart) by the physician to position the AV guide over the vein and, in some embodiments, over a portion of the artery as well.
(20) As the vein matures (expands in diameter) and comes in contact with the AV guide, the edges of the slot or slit will be forced to move away from each other (the diameters of AV guide will increase) in a manner that accommodates a range of mature vein diameters within the AV guide. In other embodiments, there is little change in the distance between edges of the slot or slit and the diameters of the mature vein are limited by the initial shape of the AV guide.
(21) For a tapered tube or tubular body of the AV guide, the distal end diameter Dd and proximal end diameter Dp are related as follows: Dp<Dd where the ratio Dd/Dp may be about 2, 3, 4, or about 5, or between about 2-5.
(22) The AV guide is sized to initially fit the vein loosely and has an internal diameter in the range of 4-8 mm; the AV guide would have an ID at one or both of the proximal and distal ends of about 6 mm; the AV guide may have an internal diameter of 4-10 mm and a length of 5-10 cm; rings and links that allow the tube or tubular portion to take on multiple shapes from linear to curved.
(23) A radius of curvature for the inner side may range from about 3-5 mm and with or without a pre-made taper (i.e., larger diameter at distal end than proximal end);
(24) The AV guide is made partially or entirely from a polymer composition that permits part of, or the entire AV guide to degrade over a 4-8 week period; examples include polycaptrolactone or 50:50 PLGA-PLLA.
(25) A distal and/or proximal end of the AV guide may have curled or flared ends to minimize trauma to the vein and/or artery. When the AV guide includes a tubular body with a slot the edges of the slot may also be curled or flared outward for the same reason.
(26) A cuff is formed integrally with the tubular body or adapted for being joined to a proximal end of the tubular body.
(27) An AV fistula kit including a cuff or fingers portion configured for being placed over an artery portion, a tubular portion configured for being placed over a vein portion. The cuff and tubular portions may be separate pieces or integral with each other. The cuff and tubular portions may be configured for being sutured together.
(28) An edge adapted for receiving the artery portion of the fistula and shaped according to at least a portion of an outer surface of the artery at the fistula.
(29) An AV guide including a tubular body having variable cell sizes to accommodate a dialysis needle with low probability of striking the needle, while providing high surface coverage for drug-elution from a luminal surface thereof, e.g., at a proximal end of the AV guide, is shown and described in one or more of the embodiments in US20070142897.
(30) The AV guide's tubular body is made from a shape memory alloy including nitinol, elgiloy, or strain hardened stainless steel, and/or polymer compositions including silicones, polyurethanes, and/or fluorinated and flexible polymers including Polyvinylidene Fluoride (PVDF) or poly(vinylidene fluoride-co-hexafluoropropylene) (PVDF-HFP).
(31) A range of radius of curvature of an outer side of a tubular portion of an AV guide, expressed in terms of a distal end of the tubular portion and radius of curvature of the inner side, is ½ (2ri+Dd)≦ro≦2 (2ri+Dd), ½ (2ri+Dd)≦ro, or ro is about equal to (2ri+Dd).
(32) The AV guide may be drug-eluting. The delivered drugs can help control and shape vein maturation (expansion) dimensions to help avoid the creation of wall regions of low shear stress and thus, help prevent stenosis and fistula failure. Therapeutic agents may be incorporated into the device in a monolithic fashion or by be affixed via a coating.
(33) The AV guide has disposed on its luminal surface a composition including everolimus, sirolimus, paclitaxel or other anti-restenotic drug or drug combination applied as a coating, impregnated or infused in the AV guide. The drug is located at the proximal end 20b of the tubular portion 23 and/or cuff portion 25 of the AV guide, and/or the drug is located only or substantially along about 10%/20%, 10-30% or 10-50% of the length of the tubular portion 23 including a proximal end nearest the anastomosis.
(34) The composition may include agents, in addition or in place of those listed above, metalloproteinases, elastases, collagenases or agents that promote their production.
(35) Drugs may be released in succession. First is the release of agents that promote vessel expansion and then agents that inhibit intimal thickening.
(36) Drugs can also be released regionally. A tubular portion would have a higher concentration of the composition along the inner side (luminal surface) of the tubular portion. The intimal thickening typically occurs along this surface defined by the inner radius (ri) so it can be helpful to place agents that prevent intimal thickening in greater concentration along the inner surface defined by the inner radius.
(37) An arteriovenous (AV) guide including a tubular body having a proximal end and a distal end, the tubular body being configured to receive a vein portion of an AV fistula; a cuff or edge configured to mate with, or formed integrally with the proximal end and configured to partially or fully surround an artery portion of an AV fistula; the tubular body is both curved and tapered, wherein—a diameter at the distal end is larger than a diameter at the proximal end,—a curvature along an inner side is larger than a curvature along an outer side, and—at the inner side adjacent the proximal end an entrance angle is less than about 40 degrees as measured with respect to an axis extending across the cuff, the edge or a proximal end opening of the tubular body.
(38) The aspects of disclosure as set forth in (36), (38) or (40)-(42), in combination with one of, more than one of, or any combination in any order of the following list of things: wherein the cuff is fingers formed by a ring element at the proximal end; wherein the edge is petals, a beveled edge or a curled or flared edge; wherein a first cross-section at the proximal end taken along a plane perpendicular to a bore axis of the tubular body at the proximal end is between 80 and 90 degrees orientated with respect a second cross-section at the distal end taken along a plane perpendicular to the bore axis at the distal end of the tubular body; wherein the tubular body is made from Nitinol, a polymer composition or a combination thereof, and the tubular body comprises rings interconnected by links; wherein the tubular body is at least partially woven using a metal or metal alloy threads, a biodegradable polymer, or a combination thereof; wherein the tubular body is a wrap having a shape memory to form the tubular body, the wrap having a slot extending along an outer side of the tubular body; wherein the slot has a curvilinear form such that edges of the slot nest together; wherein a radius of the curvature along the outer side (ro) is related to a radius of the curvature along the inner side (ri) as follows: ½ (2ri+Dd)≦ro≦2 (2ri+Dd), ½ (2ri+Dd) ro, or ro is about equal to (2ri+Dd) where Dd is a diameter of the guide at the distal end; and/or wherein an inner diameter of the tubular body adjacent the distal end is 2, 4, 4, about 4-5, about 5, or between about 2-5 times larger than an inner diameter of the tubular body adjacent the proximal end.
(39) An implantable medical device, including a tubular body having a proximal end and a distal end, wherein—the tubular body has a curvature,—the curvature at an inner side of the proximal end is less than about 40 degrees, and—the curvature along the inner side is greater than a curvature along an outer side.
(40) The aspects of disclosure as set forth in (36), (38) or (40)-(42), in combination with one of, more than one of, or any combination in any order of the following list of things: wherein the tubular body is frusto-conical and has a curvature in one plane, such that an opening of the tubular body at the distal end is larger than an opening at the proximal end; wherein the tubular body has a constant diameter over at least a portion of the length of the tubular body; wherein the inner side curvature is a constant curvature and the outer side curvature varies over the length of the tubular body; wherein the inner side curvature traces about half the circumference of a circle; wherein the tubular body includes a slot extending from the proximal end to a distal end thereof, the slot being located on the outer side of the tubular body, wherein the slot forms first and second portions of the tubular body joined along the inner side by a living hinge; further including a cuff or edge integral with, or adapted to be disposed adjacent to the tubular body proximal end; wherein the cuff partially or fully circumscribes a cylindrical space orientated everywhere perpendicular to the lumen of the tubular body; wherein the tubular body comprises a network of interconnected elements or woven threads; and/or wherein a cuff or edge of the guide is integral with, or separate from a tubular portion.
(41) A method of supporting an arteriovenous (AV) fistula, including placing an tubular body over a vein, wherein the tubular body is curved, and placing a proximal end of the tubular body adjacent an artery portion of fistula.
(42) A method of making an arteriovenous (AV) guide, including selecting a separation distance d1 between a vein and artery; selecting a diameter d2 of the vein; and making a tubular body having an inner side length equal to about π*(d1+d2)/2−(0.7)*d2.
(43) A kit for making extravascular medical device, including a tubular portion for being placed over a first vessel portion; and a cuff or fingers portion for being placed over a second vessel portion; wherein when implanted the medical device substantially surrounds an anastomosis comprising the first vessel portion and the second vessel portion.
(44) The aspects of disclosure as set forth in (36), (38) or (40)-(42), in combination with one of, more than one of, or any combination in any order of the following list of things: further including a fabric and or porous cover; further including sutures for securing the medical device to tissue adjacent the anastomosis; and/or wherein the cuff and tubular portions are integral with each or separate from each other and configured for being connected to each other when implanted in a body.

### INCORPORATION BY REFERENCE

All publications and patent applications mentioned in the present specification are herein incorporated by reference to the same extent as if each individual publication or patent application was specifically and individually indicated to be incorporated by reference. To the extent there are any inconsistent usages of words and/or phrases between an incorporated publication or patent and the present specification, these words and/or phrases will have a meaning that is consistent with the manner in which they are used in the present specification.

### BRIEF DESCRIPTION OF THE DRAWINGS

FIG. 1 is a side-view of the arm of a patient receiving dialysis. A fistula is shown.

FIGS. 2A-2C show steps for forming a fistula.

FIG. 2D shows a schematic of embodiments of an arteriovenous (AV) guide for the fistula.

FIG. 2E is a cross-sectional side view of the AV guide of FIG. 2D with proximal and distal ends thereof being flared or curled outward.

FIG. 3 shows a side-view of another embodiment of an AV guide.

FIGS. 3A-3D show perspective, front, rear and top views of the AV guide of FIG. 3 having a tube configured for receiving the vein portion of the fistula. The AV guide further includes a cuff or fingers for receiving the artery portion of the fistula. FIG. 3B is taken from section B-B in FIG. 3. FIG. 3C is taken from section C-C in FIG. 3. And FIG. 3D is taken from section D-D in FIG. 3.

FIG. 4 shows a side-view of another embodiment of an AV guide. In contrast to the AV guide of FIG. 3, this embodiment has a slot along an outer side of the AV guide. The slit may be sinusoidal, undulating, or straight.

FIGS. 4A-4D show perspective, front, rear and top views of the AV guide of FIG. 4 having a tubular portion configured for receiving the vein portion of the fistula. The AV guide further includes a cuff or fingers for receiving the artery portion of the fistula. IN this embodiment a slit or slot is straight. FIG. 4B is taken from section B-B in FIG. 4. FIG. 4C is taken from section C-C in FIG. 4. And FIG. 4D is taken from section D-D in FIG. 4.

FIGS. 5A-5C show front, rear and top views of the AV guide of FIG. 4. The AV guide of FIGS. 5A-5C has a slot or slit that takes a curvilinear form, e.g., wavy, sinusoidal or undulating slit or slot as opposed to a straight slit or slot, or a non-curvilinear form, as shown in FIGS. 4A-4D. FIG. 5A is taken from section B-B in FIG. 4. FIG. 5B is taken from section C-C in FIG. 5. And FIG. 5C is taken from section D-D in FIG. 4. The slit or slot having a curvilinear form is configured so that edges nest within each other. It is believed that with nesting edges a vein portion will grow to be more circular in cross-section when it engages the outer side where the slit or slot is found.

FIG. 6 shows an embodiment of a tube or tubular portion of an AV guide cut from a tube and forming rings interconnected by links. The struts and links are made from, e. g, a super-elastic metal.

FIG. 7 shows an embodiment of a tube or tubular portion of an AV guide formed by woven threads.

FIG. 8 shows another embodiment of a tube or tubular portion of an AV guide having rings interconnected by links. Additionally there are petals at a proximal end adapted for contacting the artery portion of the fistula.

FIG. 9 shows another embodiment of a tube or tubular portion of an AV guide having rings interconnected by links. Additionally there is a closed off or beveled proximal edge to reduce trauma to the artery portion of the fistula.

FIG. 10 shows another embodiment of a tube or tubular portion of an AV guide having rings interconnected by links. Additionally there are fingers at a proximal end adapted for being wrapped around the artery portion of the fistula.

FIG. 11 shows another embodiment of a tube or tubular portion of an AV guide made from a polymer tube.

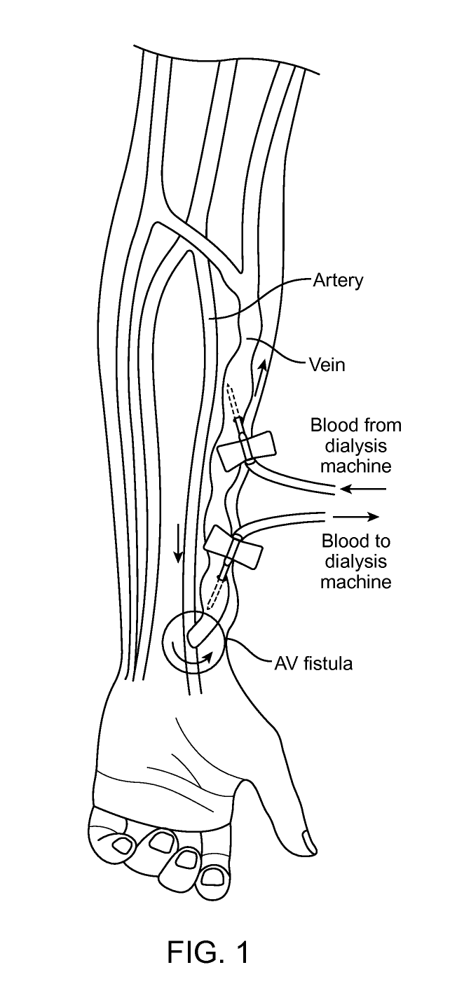

### DETAILED DESCRIPTION OF EMBODIMENTS

For purposes of this disclosure, the following terms and definitions apply:

When referring to a vein or artery prior to making a fistula, a “proximal end” refers to an end closest to the torso of the body, whereas a “distal end” refers to the end furthest from the torso of the body. In contrast, after the fistula is made, when referring to a medical device's intended location relative to a fistula or anastomosis, the terms “proximal” and “distal” are instead made with respect to the relative location of the fistula or anastomosis. Thus, for example, the end of a scaffold closest to the fistula will be called the “proximal” end and the end furthest from the fistula the “distal” end. Thus, generally speaking, prior to making the fistula the former terminology is used. And after the fistula is made “proximal” and “distal” always refers to a location relative to the fistula.

A “curvilinear form”, in reference to a description of a shape of a space, or nature by which edges of a tubular portion of an AV guide nest together, means a form consisting of or bounded by curved lines, represented by a curved line. Examples include a curvilinear form made by a edges that are sinusoidal, undulating or wavy.

The terms “anastomosis” and “fistula” may be used interchangeably in this description. For purposes of the disclosure the two terms mean the same thing and refer to the arteriovenous (AV) type of anastomosis or fistula.

The term “about” means 20%, 15%, 10%, 5%, 4%, 3%, 2%, 1.5%, 1%, between 1-2%, 1-3%, 1-5%, or 0.5%-5% less or more than, less than, or more than a stated value, a range or each endpoint of a stated range, or a one-sigma, two-sigma, three-sigma variation from a stated mean or expected value (Gaussian distribution). It is understood that any numerical value, range, or either range endpoint (including, e.g., “about none”, “about all”, etc.) preceded by the word “about” in this disclosure also describes or discloses the same numerical value, range, or either range endpoint not preceded by the word “about”.

A “stent” is a permanent structure, usually comprised of a metal or metal alloy, generally speaking, while a scaffold will refer to a structure comprising a bioresorbable polymer and capable of radially supporting a vessel for a limited period of time, e.g., 3, 4, 6 or 12 months following implantation. It is understood, however, that the art sometimes uses the term “stent” when referring to either type of structure.

An AV fistula is usually first created near the wrist of a patient. A brief description of an idealized and preferred procedure for forming the fistula follows.

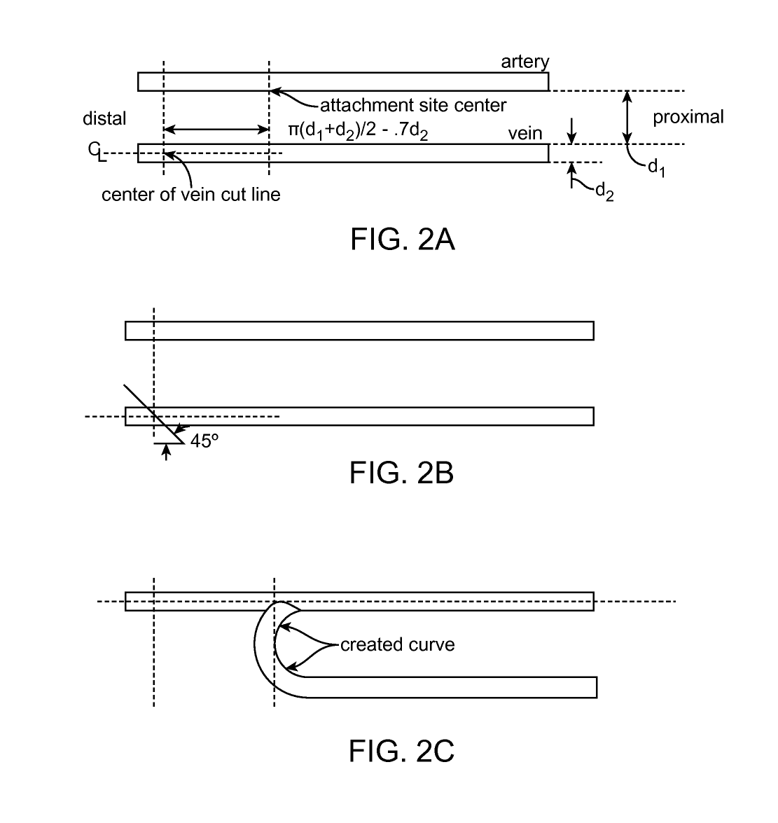

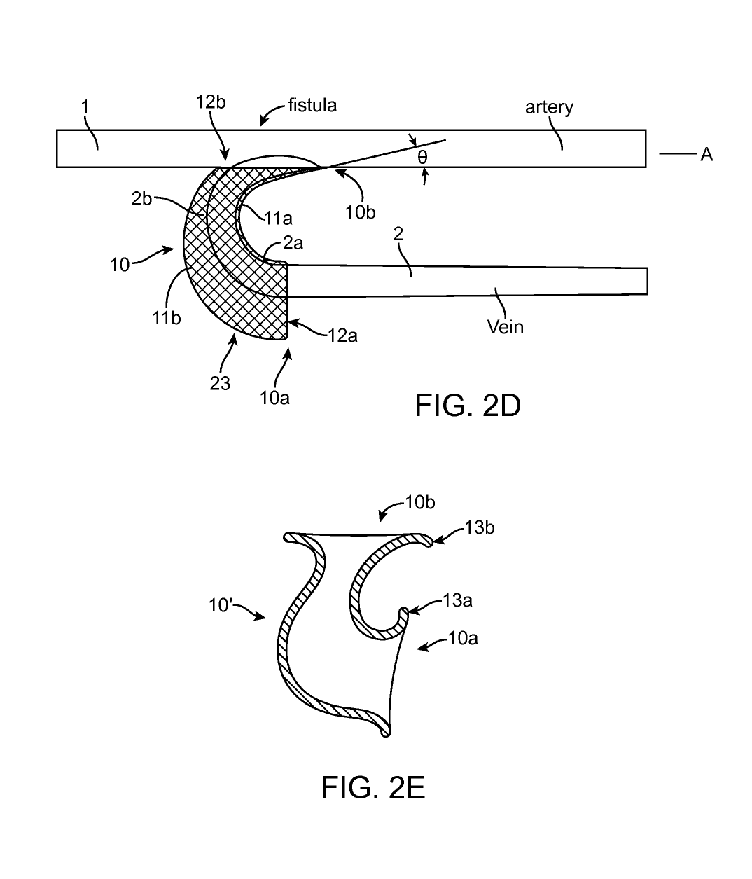

Referring to FIGS. 2A-2C, a vein and artery pair that is not a collateral pair is chosen for making the fistula. The chosen vein and artery preferably have a significant separation from each other. A measurement is made of the separation distance “d1” (i.e. artery to vein inside) and the vein diameter “d2”.

Next, an appropriate distal site at which the vein will be cut and a more proximal site where the proximal vein will be surgically attached to the artery is found. If the separation of vein and artery near the attachment site is “d1” and the vein diameter is “d2”, then choose the attachment site center on the artery to be about “π (d1+d2)−0.7(d2)” proximal of the center of the chosen vein cut line.

A brief description of the procedure follows. Cut the vein at about a 45° angle. Then, using standard surgical procedures, shape the vein end and attach the vein end over a created opening in the artery wall that is centered at the attachment site center. Thus, the vein is attached such that it may form a curved shape and blood flow into the vein may more gradually change its flow direction as it flows through the created curve. This attachment shape allows the highest flow rates of blood into the vein and thus, improves the chances of the AV Fistula maturing properly.

As the vein matures (increases in diameter to a final diameter), there is no guarantee that the vein will retain the flow facilitating curve shown in FIG. 2C. Thus, an extravascular or perivascular arteriovenous (AV) guide may be placed over the vein at and near the anastomosis (attachment) site to support and help achieve vein maturation in the desired curve shape. According to the disclosure this AV guide is configured to maintain a curved shaped for the vein portion of the fistula as it expands in diameter during the maturing process. As mentioned earlier, when the vein portion of the fistula has a curved shape, there occurs a higher flow rate into and/or through the vein portion than if the vein were allowed to remain straight nearest the fistula. The higher flow rate supports the desired maturing process and causes the flow characteristics/patterns of this section of the matured vein to be such that there are no regions of low wall shear stress and/or less circular/stagnant flow along in the vein wall, which helps prevent a stenosis from forming at the fistula or adjacent portions of the vein. Preferably the AV guide is such that it causes the vein to mature into a shape producing a relatively low acceleration (rate of direction change) of the flow as it is diverted from the artery to vein. Moreover, the shape minimizes or eliminates stagnant or circular blood flow and avoids the forming of low flow regions that result in minimal or no shear stress along the vessel walls.

A preferred support structure will be curved, have an ID that is configured to increase distally as the vein matures and grows in diameter or, and more preferably, has a pre-made and/or as-implanted tapered shape so that the diameter increases distally of the proximal end of the AV guide. In these embodiments the AV guide allows for, and/or guides the expansion of the vein into the desired curved shape as the vein matures. A AV guide with relatively smaller proximal ID limits vein expansion at the anastomosis site and thus, limits artery damage at the anastomosis site due to vein expansion. With less continuing expansion damage during the maturation process, the anastomosis site may be less prone to stenosis. It is preferred that this extravascular AV guide is designed such that it may be positioned after the anastomosis is made. If the AV guide is designed to be placed over the vein before the anastomosis is made, the AV guide may interfere with the anastomosis procedure. In a less preferred embodiment, the AV guide may be placed or positioned over the vein proximally, prior to forming the fistula. Then, after the fistula is formed the extravascular support is moved toward the anastomosis site and positioned relative to the fistula as desired.

In some embodiments, the AV guide is initially held in its final position with reabsorbable sutures (i.e. cat gut sutures) that connect the AV guide to nearby fascia or tissue adjacent the vein. In some embodiments, the AV guide includes holes, eyelets or other structural features that accommodate sutures (not shown). Tissue in-growth supports the implant after a period of time.

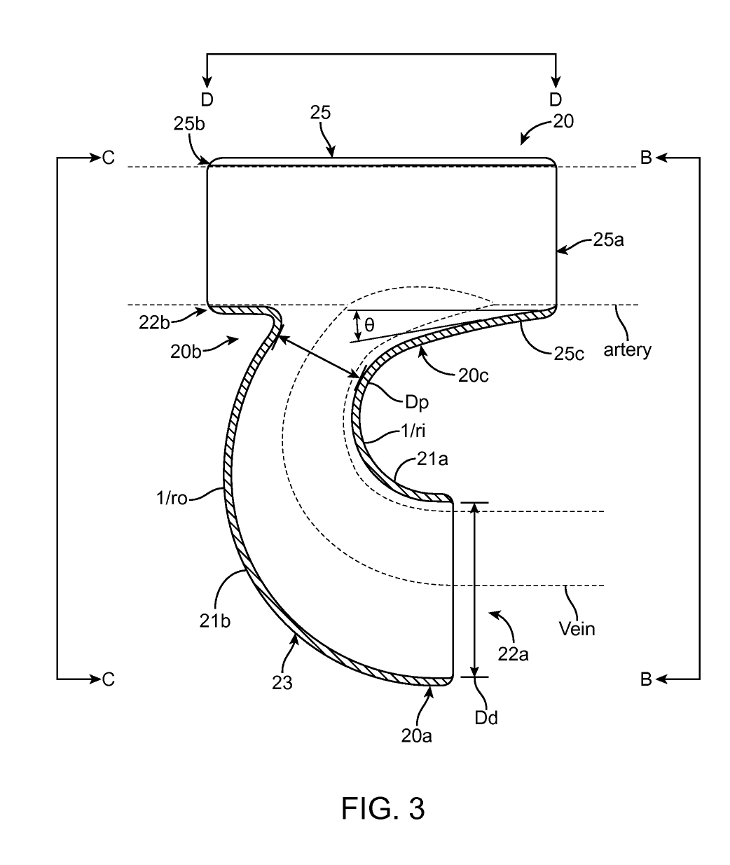

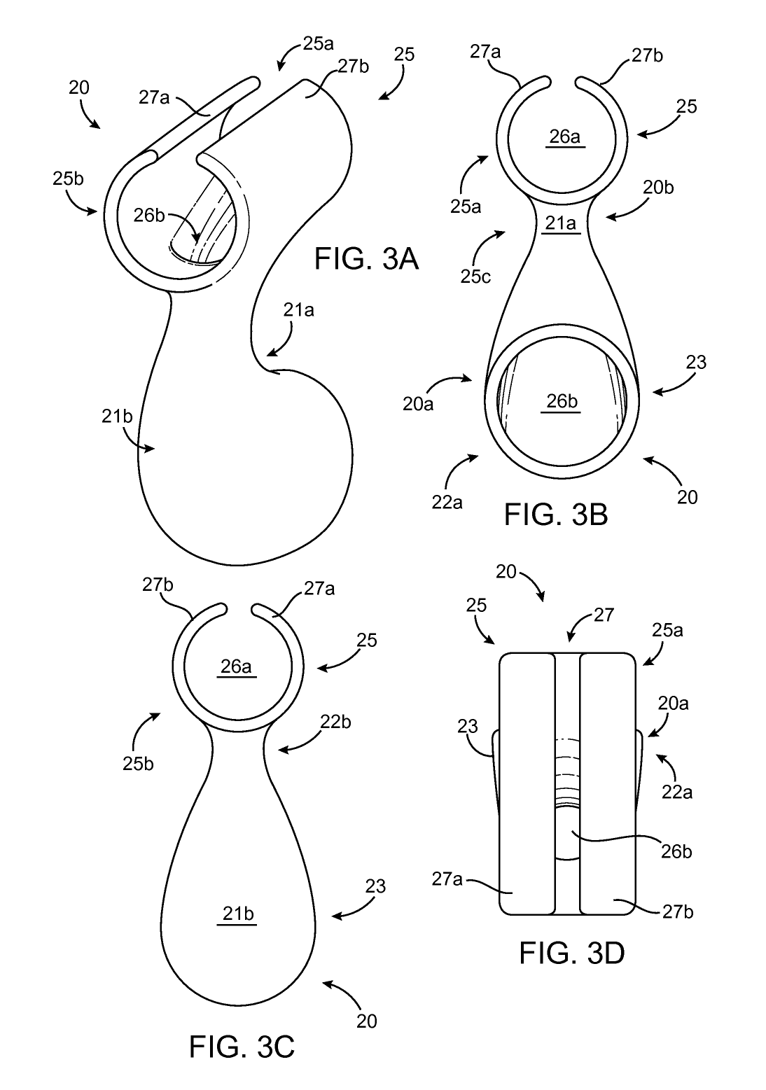

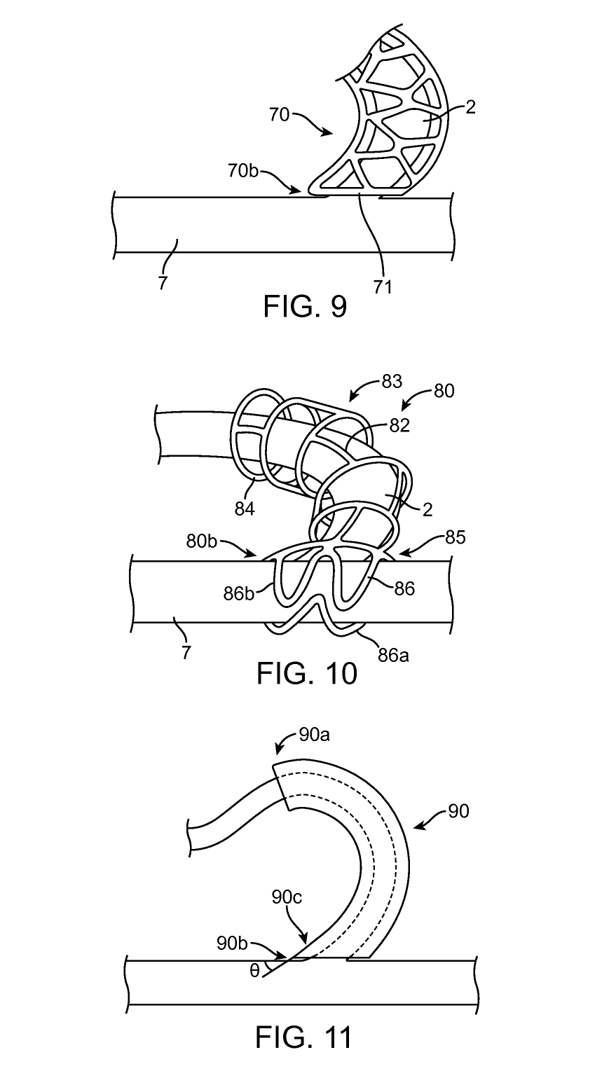

In some embodiments the proximal end of the AV guide has structure that extends over at least a portion of the OD of the artery to help secure the implant into position and to support any parts of the vein or its attachment site that may extend over the OD of the artery (shown). An example of such structure may be a cuff 25 (FIG. 3) having fingers 27a, 27b (FIG. 3A) or fingers 86A, 86b (FIG. 9) configured to partially or fully circumscribe the artery proximal the fistula.

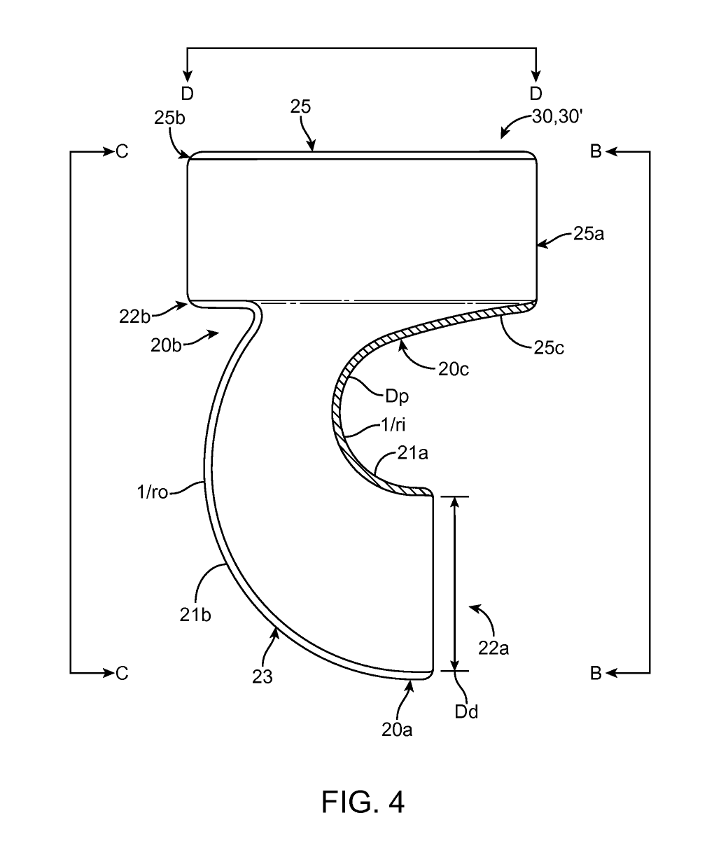

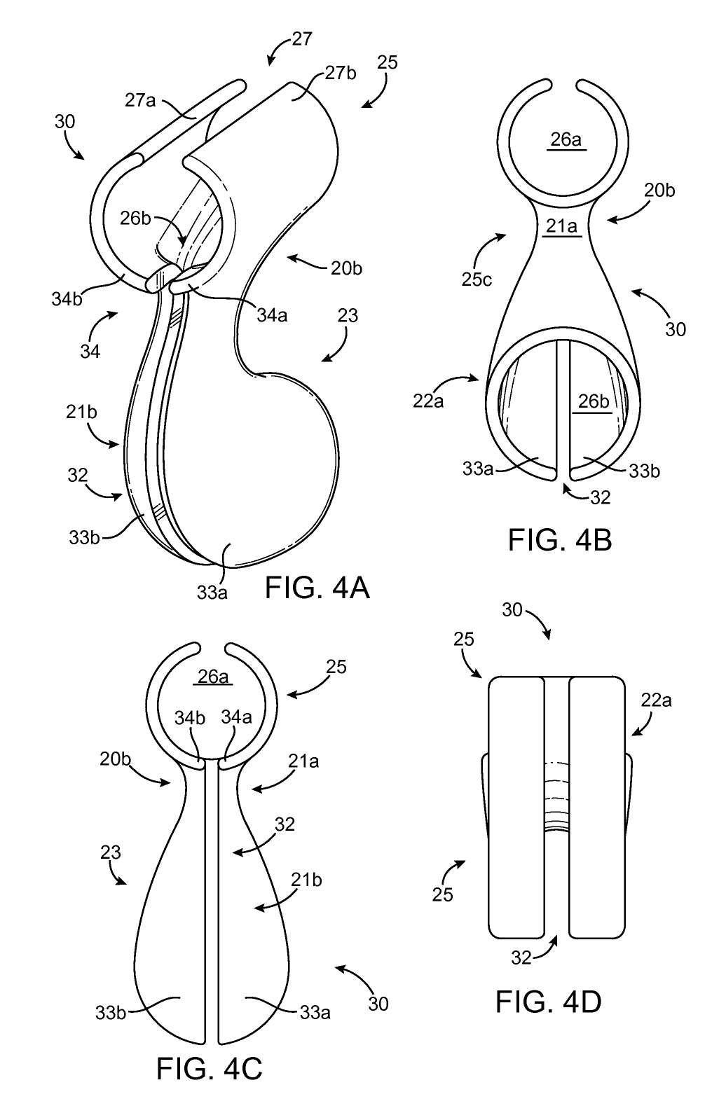

In some embodiments proximal and/or distal ends (e.g., ends 13a, 13b in FIG. 2E), or longitudinal seams or edges (e.g., edges 34a, 34b, 34a, 34b in FIGS. 4A-4D) of the AV guide may be curled or flared outward to make them less traumatic to an expanding (maturing) vein. The flared edges and/or ends may be formed after the AV guide is formed or incorporated when the piece is formed. In some embodiments the proximal end 10b of the AV guide 10 may have an edge that reduces trauma when pressed into the artery during placement (e.g., beveled edge 71 of FIG. 8 or petals 66 of FIG. 7).

In the embodiments an AV guide (e.g., AV guide 10 depicted in FIG. 2D or FIGS. 5-10) may be made from a bio-reabsorbable material or non-bioresorbable material. In the latter case, the AV guide material may be a shape memory alloy such as nitinol, elgiloy, or strain hardened stainless steel, e.g., as disclosed in U.S. Pat. No. 6,663,664 to Pacetti (with or without the variable radial force feature using a biodegradable polymer).

Examples of suitable polymers or compositions comprising one or more polymers for the AV guide are described in U.S. application Ser. No. 13/252,120 (attorney docket 104584.22), U.S. application Ser. No. 13/525,145 (attorney docket 104584.44) and U.S. application Ser. No. 13/584,678 (attorney docket 104584.44). Although the scaffolds discussed in those applications are intended to be used as a balloon-expandable, intravascular scaffold for supporting a vessel as an intravascular scaffold, it will be appreciated, without further explanation being necessary, that the same material and scaffold pattern(s) may be used to form at least a tubular portion of the AV guide for at least some of the embodiments, e.g., as described in FIGS. 3, 3A-3D, 4, 4A-4D, 5A-5C or FIGS. 6-11. In the embodiments the tubular, tapered or non-tapered body may be made by extrusion, blow-molding or injection molding a polymer or polymer blend. Examples of suitable AV guide flexible polymeric materials with an appropriate memory behavior are silicones, polyurethanes, and fluorinated and flexible polymers such as Polyvinylidene Fluoride (PVDF) and poly(vinylidene fluoride-co-hexafluoropropylene) (PVDF-HFP).

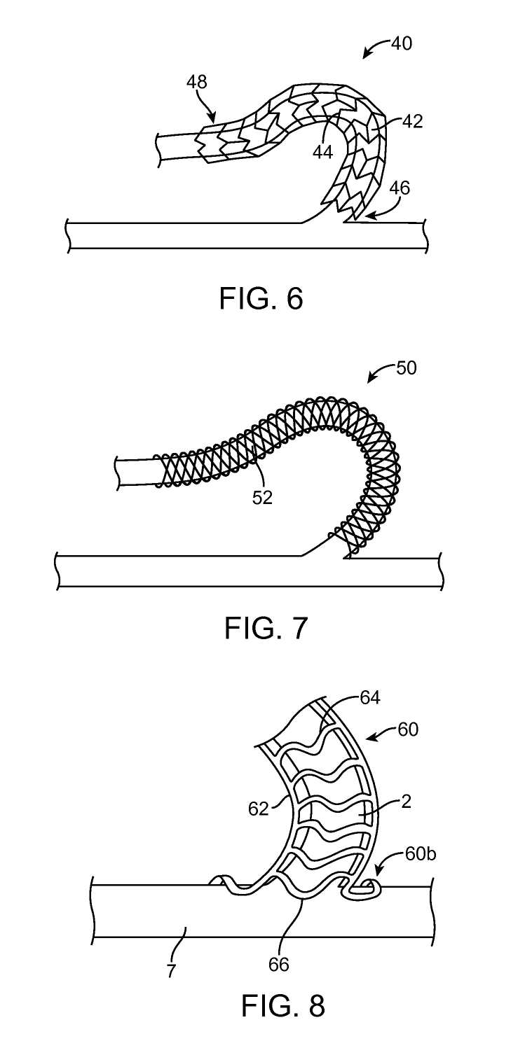

The AV guide may also be partially or entirely made as a woven body, such as in the embodiment of FIG. 6. Examples of woven stent or scaffold structures are found in U.S. Pat. No. 6,245,104 and US 2010/0298952.

The following describes several embodiments of an AV guide, including embodiments illustrated in FIGS. 2D, 2E, 3, 3A-3D, 4, 4A-4D, 5A-5C and FIGS. 6-11. It is understood that the features of these separate embodiments are not mutually exclusive of each other. One of ordinary skill will appreciate that the various features of these embodiments may be combined in different ways to produce AV guides in other embodiments without departing from the scope of the disclosure.

FIG. 2D illustrates in schematic a fistula where an artery 1 and vein 2 are joined. An AV guide 10 is disposed over the proximal vein portion 2a, so as to function as an extravascular stent (or scaffold) for the vein portion 2a. The scaffold 10 is pre-shaped as a curved, tubular body to encourage or cause the vein portion 2a to assume a curved shape proximal the fistula during the maturation process. The AV guide 10 has an inner curved part 11a and an outer curved part 11b, each of which extend from a distal end 10a to a proximal end 10b of the AV guide 10 (“inner” and “outer” parts may be understood as the A V guide parts nearer the vein portion downstream and upstream of the fistula, respectively. The same terminology is adopted for outer and inner surfaces/sides of the AV guide). The AV guide 10 is preferably both curved and tapered, having a smaller diameter at the proximal end 10b than a diameter at the distal end 10a.

The inner and outer side curvatures of the AV guide 10 may depend on the size of the fistula and/or distance between the vein and artery portions (distance d1 in FIG. 2A). The curvatures will also depend upon whether the scaffold 10 is formed to provide direct support along the inner side 2a, the outer side 2b or both as the vein matures. The sides 11a, 11b may be therefore shaped accordingly to this criterion (either prior to forming the fistula or after the fistula is formed by making appropriate adjustments when the AV guide is being sutured to adjacent fascia—it will be appreciated that the AV guide may be made to have a curvature that can be adjusted by the surgeon when it is being tethered to nearby tissue in the body to achieve the desired curvature or guide at the inner side). In FIG. 2D the inner side 11a is shaped to provide direct support for the inner side 2a of the vein at the time AV guide 10 is implanted, while the outer curved part 11b provides space for the vein to expand out, as shown. Thus, at the time of implantation only the inner side 11a guides the growth of the vein. However, after the vein's diameter has enlarged enough to contact the outer side 11b, the outer side 11b may also guide the growth of the vein as it continues to mature.

Thus, for the AV guide 10 in FIG. 2D the inside 2a of the curve in the vein portion 2 is supported at implantation by the AV guide 10 inner side 11a and the outside 2b of the created curve is not, at least initially, supported by the outer side 11b. The outside 2b of the vein portion 2 is allowed to expand freely while the inner side 2a growth is guided by the side 11a of the AV guide 10 being in contact with the inner side 2a from the time of implantation. Eventually the vein portion 2 expands enough (due to the influence of higher arterial pressure) to cause the side 2b to come into contact with the curved outer side 11b of the AV guide 10. Once the portion 2b of the vein touches or comes in contact with the side 11b, the vein's expansion may be partially restrained, or not restrained at all. At the same time the AV guide 10 deforms, primarily by an increase in diameter.

In some embodiments the space between the outer side 11b and side 2b is large enough so that there is little, if any influence on vein maturation. In those embodiments the diameter of the AV guide 10 may be sufficiently large that contact with the outer side 2b occurs, if at all, only after the vein has reached about its largest diameter. In some embodiments the AV guide 10 is formed as a perivascular wrap having a slot or open seam along its length (as opposed to a tubular body). Thus, in some embodiments that use a perivascular wrap the AV guide 10 may have all or a portion of the outside 11b of the curve as an open/unsupported region/slot so that the AV guide 10 is capable of being easily pushed open during the vein portion 2 maturation process.

In accordance with one or more of the foregoing, the outer curved part 11b may have variable curvature between distal and proximal ends of the AV guide. For example, the outer curved part 11b may have less curvature at the proximal end 10b and distal end 10a than it has at intermediate the ends 10a, 10b. The inner curved part 11a (or at least a portion thereof) may have a constant curvature. In some embodiments the inner curved part 11a may trace about half the circumference of a circle. The outer curved part and inner curved parts 11b, 11a do not have the same curvature

The AV guide is preferably made to have a tapered shape. Distal opening 12a of the AV guide 10 has a larger opening than the proximal opening 12b. In the embodiments the inner curved part 11a has a higher curvature at the proximal end 10b than the outer curved part 11b, as it is desirable to have a low take-off angle θ. For example, the take-off angle may be about 5, 10, 15, 20, 25 or less than about 30 degrees relative to axis A in FIG. 2D. The outer part 11b (or at least a portion thereof) may have a constant curvature, although different from and less than the curvature of the inner part 11a. Stated differently, the outer part 11b may trace a portion of the circumference of a larger circle than the inner part 11a. In the embodiments having a tapered tube and constant curvatures for the outer side and inner side the centers of the respective circles (from which the radius of curvature is measured) would be offset from each other in at least the vertical direction in FIG. 2D.

The opening 12b at the proximal end 10b is orientated at about 120 to 80 degrees to the opening 12a at the distal end 10a. The distal end 10a may be extended towards the right in FIG. 2D at about the same diameter as the diameter at the opening 12a. Thus, a cylindrical shaped extension part with constant diameter Dd may extend to the right from, and be integral with the AV guide 10 of FIG. 2D.

As mentioned earlier and described in greater detail below, a cuff or fingers may be formed at the proximal end 10b. The cuff or fingers may be shaped to conform to the artery portion of the fistula, or even fully or partially wrap around the artery portion for added support, to help stabilize the AV guide position at the fistula and/or to avoid having exposed edges cause undue trauma to the adjacent maturing vein. Additionally, or alternatively proximal and distal ends 10b, 10a may be flared or curled open. In the case of a rolled, curled or flared end reference is made to the cross-sectional view of FIG. 2E, which shows proximal and distal ends 13b, 13a of the AV guide 10′ flared open so that the proximal and/or distal edges of the AV guide 10′ causes less trauma to the maturing vein.

FIGS. 3, 3A-3D, FIGS. 4, 4A-4D and FIGS. 5A-5C show embodiments of an AV guide with a cuff or fingers and with or without a slit or slot along an outer edge, respectively. An AV guide with a slit or slot may be thought of as a tubular wrap (having a tubular shape and, in cross section, a circular or C-shape when viewed in a plane perpendicular to the bore axis of the tubular wrap) and an AV guide without a slit or slot may be thought of as a tube. In both these embodiments the AV guide is curved and tapered so that the distal end has a larger opening than the proximal end.

FIGS. 3 and 4 show, respectively, a side view of embodiments of an AV Guide: a vein supporting portion is a tube-type AV guide 20 (FIG. 3), and the vein supporting portion is a tubular wrap-type AV guide 30 (FIG. 4). For both these embodiments, the AV guide 20, 30 has a distal diameter Dd and proximal diameter Dp (Dp<Dd where the ratio Dd/Dp may be about, or greater than about 2, 3, 4, or about 5, or between about 2-5). The tubular portion 23 in these embodiments has an inner side 21a with curvature 1/ri, outer side 21b with curvature 1/ro (1/ri>1/ro and either or both may have a constant curvature). The tubular portion 23 has a distal opening 22a oriented at between about 80 to 120 degrees from the proximal opening 22b.

The AV guide 20, 30 have a cuff portion 25 configured to form an inner diameter enabling it to be easily fit over the artery portion of the fistula, thereby enabling the cuff 25 to be fully, partially or substantially circumscribing the artery portion of the fistula. The cuff portion 25 is preferably integrally formed with the tubular portion 23 of the AV guide 20, 30. The cuff portion 25 includes an outer side 25b and an inner side 25a. In other embodiments the cuff may be separate from the tubular portion 23, but configured to mate with the proximal end 20b, e.g., the portions 23, 25 may be stitched or tethered to each other after placing each over the vein and artery, respectively.

The take-off angle θ is measured at the entry portion 20c of the inner side 21b, at the proximal end 20b just to the left of the leading edge of the cuff portion 25c in FIG. 3. The take-off angle θ relative to the horizontal leading edge 25c (or bore axis of the cuff portion 25) may be about 5, 10, 15, 20, 25 or less than about 30 Degrees.

Referring now to FIGS. 3A-3D there is shown respective perspective, front, rear and top views of the AV guide 20 of FIG. 3. The description of FIGS. 3A-3D applies equally to FIGS. 4A-4D unless otherwise noted. FIGS. 3A, 3A show a perspective view, FIGS. 3B, 3B show a front view taken at Section B-B in FIG. 3, FIGS. 3C, 3C show a rear view taken at Section C-C in FIG. 3, and FIGS. 3D, 3D show a top view taken at Section D-D in FIG. 3.

The cuff portion 25 has a slot 27. The semi-circular like fingers 27a, 27b forming the slotted 27 are flexible and may be easily pulled apart to allow the vein to be placed within the portion 25 after the fistula is made. Prior to forming the fistula, the guide 20 is disposed proximally over the vein with the fingers 27a, 27b drawn back so that the tubular portion 23 may be easily slide over the vein towards the fistula after it is formed. Then, once the proximal end 20b is positioned at its intended location at the fistula (see end 10b in FIG. 2D) the fingers 27a, 27b may be released to wrap around the artery, thereby enclosing the artery within the space 26a (FIG. 3B). The space for passage of the vein is 26b (FIG. 3B).

The description for the AV guide 30 shown in FIGS. 4, 4A-4D is the same as the AV guide 20 except as follows. The AV guide 30 has a slot or slit 32. As shown, the slot 32 separates both the cuff 25 and the tubular portion 23 into two portions connected along the inner sides 25a, 21 a. The slot 32 is formed by edges 33a and 33b of the tubular portion 23 and edges 34a, 34b of the cuff portion 25. The edges 34, 33 may be thought of as two portions together forming a tubular-shaped AV guide and being connected to each other through a living hinge along the inner sides 25c, 21 a. This configuration for the AV guide may be preferred over AV guide 20 for one or more of the following reasons.

Preferably the AV guides 20, 30 and 30′ are made as porous structures, e.g., they take the shapes shown in FIGS. 3, 4, and 5A-5C by structure shown in FIGS. 6-11. The AV guides 20, 30 and 30′ are preferably not solid or non-porous. Instead, they are into the illustrated shapes with highly porous or open structures forming tubular portion 23 and/or cuff portion 25.

The AV guide 30 may be placed at the fistula after the fistula is formed; that is, it need not be placed over a proximal vein portion prior to making the fistula because the slot 42 formed by edges 34a, 34b, 33a, 33b for the cuff portion 25 and tubular portion 23, respectively, enable the cuff portion 25 and tubular portion 23 to be opened up on the outer sides 25b, 21 b to place them over the artery and vein respectively. As shown the seam 32 extends from the distal opening 22a to the slot 27 formed by the fingers 27a, 27b. The tubular portion 23 may be radially elastic; that is, if stretched or deformed by application of external forces, then those forces are removed, the tubular portion 23 will return to the shape illustrated in FIGS. 4A-4D. The tubular portion 23 may have a range of radial elasticity such that the diameter Dp or Dp may be radially expanded to about 2-5 times its un-deformed diameter then when the external load is removed it returns to its original diameter Dp, Dd, i.e., little or no plastic deformation occurs.

Thus, according to some of the embodiments disclosed, an AV guide 10, 10′, 20 or 30 may therefore be regarded as a guide to maintain a curved shape in the vein portion 2 of the fistula, yet not unduly restrict growth in diameter of the vein portion 2 during maturation. The embodiments depicted in FIGS. 2D, 2E, 3 and 4 achieve this goal as follows. The inner sides support the vein inner side 2a at the time of implant by direct contact, but the outer side is spaced away from the vein outer side 2b and/or may have any open, slotted or unsupported region along the outer side. The inner side functions to maintain the angle θ or curvature of the inner side primarily at the proximal end, while the outer side, being spaced from the vein 2 at implantation and/or having a slot or opening, permits un-hindered radial expansion of the vein 2 portion, at least during a portion of the maturation process. In this way the desired curve in the vein portion 2 forms during maturation, yet the vein portion 2 radial expansion is not restrained in such a manner as to cause trauma.

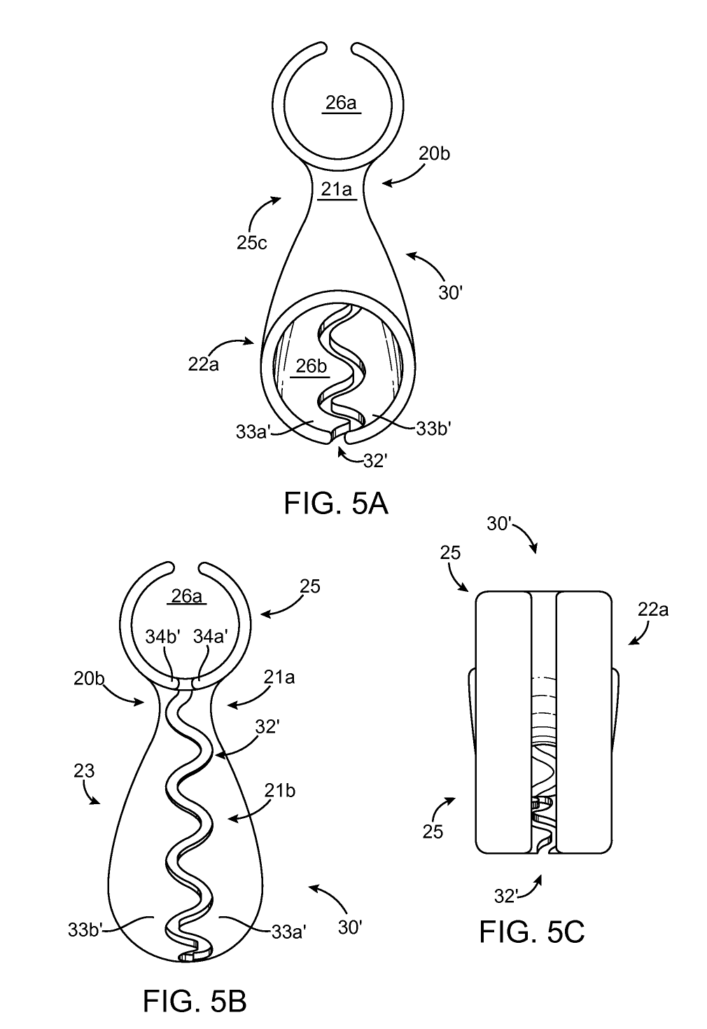

FIGS. 5A-5C show respective front, rear and top views of a more preferred embodiment of an AV guide 30′. The AV guide 30′ shown is the same as the AV guide 30 of FIGS. 4A-4D except, rather than have a straight slit or slot 32 the AV guide 30′ has a slot 32′ that takes a curvilinear form characterized by opposed edges 33a′, 33b′ that nest together. For example the curvilinear form may describe a space that is wavy, sinusoidal, undulating or zig-zag. It is believed that a slot taking a curvilinear form will better promote a circular cross-section of the vein portion disposed within the tubular portion 23 during maturation than a slot that is straight.

In other embodiments the AV guide outer side may be designed to support the outside of the vein portion 2b as well. However, this is less preferred because this may adversely affect the curvature at the inner side or traumatize the vein maturation process in the presence of increase arterial pressure.

With respect to a design, or method of design, the implant design and dimensions (model number) may be selected based on the values of d1 and d2 (FIG. 2) and/or other physiologic measurements made prior to, or at the beginning of, or during the procedure For example, if the diameter of the vein (d₂) were 4 mm, then the proximal diameter of the AV guide (Dₚ) may be chosen to be 4.5 mm (or more generally 5-15% greater than the artery diameter) to allow some expansion of the anastomosis site to compensate for the stenosis due to suture injury, but to limit the expansion such that the suture site may heal more quickly and not be subject to additional damage (expansion damage) during vein maturation that could cause a significant stenosis. Similarly, the distal diameter of the AV guide (D_{d}) may be chosen to be about 2, 3, 4, 5, greater than about 2, or between 2-5 times greater than the diameter of the vein (d₂), e.g., 8 mm, to accommodate the expected diameter of the matured vein.

The inside radius of curvature of the AV guide (i.e., 1/ri in FIG. 4) may be chosen to be about one half of the distance between the vein and the artery (d₁). In some cases, were this radius is too small to provide the desired benefit, a minimum radius needed to supply the benefit is supplied and d₁ is used to specify the length (or arc of curvature) of the AV guide required such that the distal diameter of the AV guide (D_{d}) may be at the proper angle and distance from the artery to place the vein inside the distal diameter without lateral pressure on the vein (without causing the vein to bend at an angle/without causing the vein to not be very straight). In such a manner, these or other measurements of the proposed site of the fistula may be used to specify the particular design of the AV guide that is recommended for that specific fistula site.

Referring again to FIGS. In other embodiments, a radius of curvature for the inner curve (ri) can be equal to about d½—the distance between the artery and vein (d1) divided by 2. And a range of radius of curvatures for the outer curve (ro), expressed in terms of the inner radius of curvature (ri) and the diameter of the distal end 22b of the tubular portion 23 can be expressed as shown in EQS. 1 or 2.

½(2ri+Dd)≦ro≦2(2ri+Dd) EQ.1

ro is about equal to (2ri+Dd) EQ. 2

Thus, for example, if a distance between vein and artery is 10 mm, and a distal diameter of the tubular portion is 5 mm, EQ. 1 yields ro as being between 12.5 and 50 mm and EQ. 2 yields ro being about 25 mm.

The AV guide may be drug-eluting. The delivered drugs can help control and shape vein maturation (expansion) dimensions to help avoid the creation of wall regions of low shear stress and thus, help prevent stenosis and fistula failure. Therapeutic agents may be incorporated into the device in a monolithic fashion or by be affixed via a coating.

According to another aspect of the disclosure the AV guide has a luminal and abluminal surface, the luminal surface configured for coming into contact with an abluminal surface of a vein and or artery portion of an AV fistula when the AV guide is placed at the fistula. The AV guide has disposed on its luminal surface a composition including everolimus, sirolimus, paclitaxel or other anti-restenotic drug or drug combination applied as a coating, impregnated or infused in the AV guide. The drug is located at the proximal end 20b of the tubular portion 23 and/or cuff portion 25 of the AV guide, and/or the drug is located only or substantially along about 10%/20%, 10-30% or 10-50% of the length of the tubular portion 23 including a proximal end nearest the anastomosis. By placing the anti-stenotic drug or drug combination on the AV guide, especially surrounding an anastomosis/suture site can help prevent stenosis and thus, prevent failure of the vein to mature. Low flow at the suture site or within vein inside the AV guide means low flow/pressure in the rest of the vein and thus, it will not mature.

The selections of drugs and release rates can be different, designed to effect positive remodeling/expansion of the vein. The agents may include, in addition or in place of those listed above, metalloproteinases, elastases, collagenases or agents that promote their production. Additionally, drugs may be released in succession. First is the release of agents that promote vessel expansion and then agents that inhibit intimal thickening. Drugs can also be released regionally. For instance, a tubular portion would have a higher concentration of the composition along the inner side (luminal surface) of the tubular portion. The intimal thickening typically occurs along this surface defined by the inner radius (ri) so it can be helpful to place agents that prevent intimal thickening in greater concentration along the inner surface defined by the inner radius.

In some embodiments the AV guide may not provide for a distally increasing ID (not shown) at implantation. In those embodiments an AV guide can have a constant diameter throughout, which is adapted to increase in size as the vein portion 2 begins to mature. In some embodiments the AV guide 10 may be designed to expand more towards the distal end by forming a scaffold having a distal end formed by biodegrading portions that degrade more quickly than biodegrading portions at the proximal end, or where the distal end has a biodegrading distal portion and the proximal end a non-degrading proximal portion.

According to this aspect of the disclosure a tubular portion 23 is made from a tube or sheet having a thickness that various over the length, so that when formed into a tubular portion the thickness of struts or members forming the tubular body will have different size cross-sections between the proximal and distal ends. Thus, for structure as described in FIGS. 6-10 the struts or links at the distal end having a smaller cross-section than struts and links at the proximal end. As such, the distal end strength can be less than the proximal end. Or the number of struts, density of ring-type structures (i.e., less ring structures per unit length) is less at the distal end than proximal end. Additionally, or alternatively, a pattern of struts forming rings interconnected by links, the structure defining cell structures, can have larger openings nearest the distal end so that the inherent radial stiffness of the tubular portion is much less than nearer the proximal end. Thus, a distal end can be made with significantly reduced strength and/or stiffness at the distal end compared with the proximal end to not unduly restrict radial growth of the vein despite the diameter of eth tubular portion being constant from proximal to distal ends. Additionally, if a cover (porous membrane/fabric) is used with the AV guide, then the cover can be attached looser distally and when struts have degraded (after providing the desired shaping). The cover can provide a restraint on vein expansion in the desired tapered fashion.

In other embodiments, there is a non-tapered tubular position. The struts forming rings have lower cross-sectional areas, e.g., by a factor of about 0.8, 0.5, or 0.2 than one or more links extending longitudinally form the distal to proximal ends. In these embodiments the radial strength of tubular body (provided by the rings) will be much less and/or degrade faster than the longitudinal strength of one or more links connecting rings. One example is found in an AV guide having the structure shown in FIG. 8. The longitudinal link 62 has a cross-sectional area that is greater than, e.g., about 1/0.8, 3, 2, or 5 times higher, than the cross-sectional area of struts forming ring elements 64. With this construction the vein can be relatively un-restrained radially during maturation while longitudinally this growth is guided by the one or more linking elements that become loosely connected, or disconnected from each other closer the distal end (while staying connected through a stronger ring element closer the proximal end) when the vein matures and/or material degrades.

FIGS. 6-11 illustrate additional aspects of embodiments of an AV guide according to the disclosure. It will be appreciated that each of these embodiments may incorporate some or all of the features of one or more previously described embodiments. For example, in the illustrated embodiments in FIGS. 6-11 all show an AV guide tubular portion 23 without a taper. That is, a distal end 10a is shown to have about the same diameter as a proximal end 10b of the AV guide tubular portion 23. It will be understood, however, that the disclosure encompasses the same network of interconnected elements cut from tubes, woven tubular bodies or tubes, but with it forming a tapered tubular body as in the schematic of FIG. 2D.

Additionally, the embodiments of FIGS. 6-11 may be understood to illustrate several embodiments of structure for forming the AV guide cuff portion 25 or tubular portion 23 (or both), e.g., cutting from a tube or forming in a mold networks of rings connected by struts and having a cuff portion and tubular portion as previously described with the tubular portion having a taper.

FIG. 6 shows a metallic stent-like tube 40 which is laser cut from a metal hypotube. The material of this stent would be a shape memory alloy such as nitinol, elgiloy, or strain hardened stainless steel. The AV guide is preformed to the desired shaped curvature during manufacturing. FIG. 6 shows this embodiment installed on a newly formed AV fistula. The stent-like body is formed by a network of link elements 42 connected to rings 44. The body 40 has a distal end 48 and proximal end 46. While the tube 40 looks like a stent, it would not be expanded, but would be implanted at the as-manufactured diameter. The metal-stent AV guide would be pre-formed to the desired shape and radius of curvature. As it has much open area, the underlying vein can still be accessed by needle puncture. The metallic struts 42, 44 themselves would not be affected by the needle puncture process, but would only deflect the needle. The cross sectional shape of the struts may be designed to facilitate deflection of a dialysis needle should one strike a strut.

Examples of tubular bodies having variable cell sizes to accommodate a dialysis needle with low probability of striking the needle, while providing high surface coverage for drug-elution, e.g., at a proximal end of the AV guide, are described in US20070142897. It is understood that the embodiments of a stent having different cell sizes and/or different concentration of drugs nearer or further form an anastomosis may be adapted for use as an extravascular device in accordance with the disclosure. The larger cell sizes would be located nearer the distal end and smaller cell sizes closer the proximal end to allow a dialysis needle to axis the lumen without striking the guide (distal end) and providing greater coverage area for an anti-stenosis drug (proximal end). Preferably the structure would have a slot so that it can be placed at the fistula after the fistula is formed.

FIG. 7 shows a tubular body 50. According to these embodiments there is a tube like in the case of tube 40. However, this one is fabricated not by laser machining a hypotube but by weaving metal wires into a mesh or woven tube. The wires 52 of the mesh have a round cross-section which would deflect a penetrating needle. The mesh of wires 52 may be woven in overlapping helical patterns.

FIG. 8 shows an embodiment of a metal or polymer AV guide 60 that includes a plurality of petals 66 at the proximal end 50b. The petals 66 are formed by a modified undulating ring 64 shaped to partially surround the artery 1 portion of the fistula. The ring 64 (as with the other rings 64 of the AV guide 60) is connected to the remaining structure of tube 60 by links 62. The petals 66 distribute the stresses against the artery more uniformly, thereby reducing trauma.

In FIG. 9 there is an embodiment of an AV guide were a proximal edge 70b of the tube 70 is rounded or squared off relative to the rest of the AV guide. This edge 71 is expected to be occasionally pressed into the artery. During movement of the arm, it is very possible that the AV guide will flex and shift around. Mechanical damage where the proximal edge of the AV guide presses against the artery is possible. One possible soluble is an AV guide where the proximal edge 71 has a bevel that allows the edge 71 to sit more squarely against the artery, thereby imposing less of a sharp or protruding edge into the artery, which can cause trauma.

The AV guide of FIG. 10 includes a tubular portion 84 and fingers 86a, 86b which partially or fully wrap around the artery 1. The tubular portion 84 is formed by rings interconnected by links 82. At the proximal end 80b, the fingers 86a, 86b may be formed by a specially formed proximal end ring 86 shaped to wrap around the artery 1. The ring 86 thus is shaped to encircle the tubular portion 84 lumen as well as partially or fully circumscribe the artery, i.e., via the fingers 86a, 86b.

The fingers 86a, 86b may be understood as a species of the cuff portions 27a, 27b of FIG. 3 or 4. Thus, the fingers 86a, 86b may be spread apart which, when released, wrap around the artery at the anastomosis. This may provide very secure fixation of the AV guide on the vein, preventing it from sliding around. The embodiment of FIG. 9 may have a slot along an outer side thereof (not shown) or it may be a tube (as shown). In the former case the rings 84 would instead be C-shaped sections with the opening coinciding with the slot along the outer side. Thus the embodiment of FIG. 10 may be understood as a species of both FIGS. 3A-3D and FIGS. 4A-4D.

FIG. 10 may also be understood as an embodiment of FIGS. 5A-5C where instead of a straight slot (FIG. 4) the slot takes a curvilinear form. This can be appreciated by the ends of the C-shaped rings being different lengths apart from ring to ring and a curvilinear or straight link element connecting ends between adjacent rings. As such a space extending from proximal end to distal end takes on a curvilinear form.

As described early in connection with FIGS. 3A-3D, installation of the AV guide of FIG. 10 would require it to be slid onto the vein after it has been surgically severed (unless it had a slot along the outer side). At the proximal end of the AV guide, the fingers would be temporarily held open with a clamp. After the vein has been anastomosed to the artery, the AV guide would be slid into position and the fingers 86a, 86b released.

FIG. 11 shows an embodiment of a tube 90 that is a polymeric tube with a pre-shaped curvature. It could be a fenestrated, or a solid tube. A solid tube of silicone would still allow needle puncture through it. The tube 90 has a proximal end 90b with take-off angle θ, and as is the case with FIGS. 6-10, may incorporate one or more of the features described in connection with FIGS. 2D, 2E, 3, 3A-3D, 4A-4D and 5A-5C.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the claims should not be construed to limit the invention to the specific embodiments disclosed in the specification. Rather, the scope of the invention is to be determined entirely by the claims, which are to be construed in accordance with established doctrines of claim interpretation.

What is claimed is:

1. An arteriovenous (AV) guide, comprising:
a tubular body having a proximal end and a distal end, the tubular body being configured to receive a vein portion of an AV fistula;
a cuff or edge configured to mate with, or formed integrally with the proximal end and configured to partially or fully surround an artery portion of an AV fistula;
the tubular body is both curved and tapered, wherein
a diameter at the distal end is larger than a diameter at the proximal end,
a curvature along an inner side is larger than a curvature along an outer side, and
at the inner side adjacent the proximal end an entrance angle is less than about 40 degrees as measured with respect to an axis extending across the cuff, the edge or a proximal end opening of the tubular body.

2. The guide of claim 1, wherein the cuff is fingers formed by a ring element at the proximal end.

3. The guide of claim 1, wherein the edge is petals, a beveled edge or a curled or flared edge.

4. The guide of claim 1, wherein a first cross-section at the proximal end taken along a plane perpendicular to a bore axis of the tubular body at the proximal end is between 80 and 90 degrees orientated with respect a second cross-section at the distal end taken along a plane perpendicular to the bore axis at the distal end of the tubular body.

5. The guide of claim 1, wherein the tubular body is made from Nitinol, a polymer composition or a combination thereof, and the tubular body comprises rings interconnected by links.

6. The guide of claim 1, wherein the tubular body is at least partially woven using a metal or metal alloy threads, a biodegradable polymer, or a combination thereof.

7. The guide of claim 1, wherein the tubular body is a wrap having a shape memory to form the tubular body, the wrap having a slot extending along an outer side of the tubular body.

8. The guide of claim 7, wherein the slot has a curvilinear form such that edges of the slot nest together.

9. The guide of claim 1, wherein a radius of the curvature along the outer side (ro) is related to a radius of the curvature along the inner side (ri) as follows: ½ (2ri+Dd)≦ro≦2 (2ri+Dd), ½ (2ri+Dd)≦ro, or ro is about equal to (2ri+Dd) where Dd is a diameter of the guide at the distal end.

10. The guide of claim 1, wherein an inner diameter of the tubular body adjacent the distal end is 2, 4, 4, about 4-5, about 5, or between about 2-5 times larger than an inner diameter of the tubular body adjacent the proximal end.

11. An implantable medical device, comprising:
a tubular body having a proximal end and a distal end, wherein
the tubular body has a curvature,
the curvature at an inner side of the proximal end is less than about 40 degrees, and
the curvature along the inner side is greater than a curvature along an outer side.

12. The medical device of claim 11, wherein the tubular body is frusto-conical and has a curvature in one plane, such that an opening of the tubular body at the distal end is larger than an opening at the proximal end.

13. The medical device of claim 11, wherein the tubular body has a constant diameter over at least a portion of the length of the tubular body.

14. The medical device of claim 11, wherein the inner side curvature is a constant curvature and the outer side curvature varies over the length of the tubular body.

15. The medical device of claim 11, wherein the inner side curvature traces about half the circumference of a circle.

16. The medical device of claim 11, wherein the tubular body includes a slot extending from the proximal end to a distal end thereof, the slot being located on the outer side of the tubular body, wherein the slot forms first and second portions of the tubular body joined along the inner side by a living hinge.

17. The medical device of claim 11, further including a cuff or edge integral with, or adapted to be disposed adjacent to the tubular body proximal end.

18. The medical device of claim 11, wherein the cuff partially or fully circumscribes a cylindrical space orientated everywhere perpendicular to the lumen of the tubular body.

19. The medical device of claim 11, wherein the tubular body comprises a network of interconnected elements or woven threads.

20. A method of supporting an arteriovenous (AV) fistula, comprising:
placing an tubular body over a vein, wherein the tubular body is curved, and
placing a proximal end of the tubular body adjacent an artery portion of fistula.
